(19) United States
(12) Patent Application Publication
(10) Pub. No.: US 2026/0103718 A1
(43) Pub. Date: Apr. 16, 2026

(54) METHODS AND COMPOSITIONS FOR SELECTIVE REGULATION OF PROTEIN EXPRESSION

(71) Applicant: Monsanto Technology LLC, St. Louis, MO (US)

(72) Inventors: Cara L. GRIFFITH, Catawissa, MO (US); Jeongwoon KIM, Chesterfield, MO (US); Brent A. O'BRIEN, St. Charles, MO (US); Heping YANG, Chesterfield, MO (US)

(21) Appl. No.: 19/111,753

(22) PCT Filed: Sep. 12, 2023
(86) PCT No.: PCT/US2023/073942

§ 371 Date: Mar. 13, 2025

Related U.S. Application Data
(60) Provisional application No. 63/375,681, filed on Sep. 14, 2022.

(51) Int. Cl. C12N 15/82; C12N 15/113

(52) U.S. Cl. CPC C12N 15/8218; C12N 15/113; C12N 15/8274; C12N 15/8279; C12N 2310/14

(57) ABSTRACT

The invention provides novel recombinant DNA, compositions, and methods for selectively regulating the expression of a transcribable polynucleotide or recombinant protein in pollen of a transgenic plant. The invention also provides transgenic plants, plant cells, plant parts, seeds, and commodity products comprising such recombinant DNA polynucleotides and compositions.

### REFERENCE TO RELATED APPLICATIONS

This application claims the benefit of U.S. provisional application No. 63/375,681, filed Sep. 14, 2022, herein incorporated by reference in its entirety.

### INCORPORATION OF SEQUENCE LISTING

The file named “MONS537WO_ST26.xml” containing a computer-readable form of the Sequence Listing was created on Aug. 17, 2023. This file is 9,811 bytes (measured in MS-Windows®), filed contemporaneously by electronic submission (using the United States Patent Office Patent Center), and incorporated by reference in its entirety.

### FIELD OF THE INVENTION

The invention relates to the field of plant molecular biology and plant genetic engineering. More specifically, the invention relates to methods and compositions for selectively regulating protein expression in the male reproductive tissue of transgenic plants.

### BACKGROUND OF THE INVENTION

Male tissue specific small interfering RNAs (mts-siRNAs) are siRNAs that selectively reduce expression of the messenger RNA (mRNA) in male tissues by their ability to guide sequence-dependent endonucleolytic cleavage of the mRNA. Mts-siRNA target elements (mts-siRNA_TE) are sequences that when operably linked to a transcribable DNA polynucleotide reduce expression of the transgene mRNA, and hence the expressed protein in male tissues through sequence-dependent endonucleolytic cleavage of the mRNA resulting from the hybridization of mts-siRNAs that are complimentary to sequences within the mts-siRNA_TE.

### SUMMARY OF THE INVENTION

Several embodiments relate to male tissue-specific small interfering RNA target elements or mts-siRNA target elements (mts-siRNA_TE). Also provided are recombinant DNA polynucleotides comprising the mts-siRNA target elements. In some embodiments, transgenic plant cells, plants, and seeds comprising the mts-siRNA target elements are provided. In one embodiment, the mts-siRNA target element is operably linked to a transcribable DNA polynucleotide. In certain embodiments, the transcribable DNA polynucleotide may be heterologous with respect to the mts-siRNA target element. Thus, an mts-siRNA target element sequence may, in particular embodiments, be defined as operably linked to a heterologous transcribable DNA polynucleotide. Several embodiments relate to methods of using the mts-siRNA target element and making and using the recombinant DNA polynucleotide comprising the mts-siRNA target element, and the transgenic plant cells, plants, and seeds comprising the mts-siRNA target elements operably linked to a transcribable DNA polynucleotide.

Thus, in one aspect, provided herein is a recombinant DNA polynucleotide comprising an mts-siRNA target element comprising a DNA sequence selected from the group consisting of: (a) a sequence with at least 90 percent sequence identity to SEQ ID NO:1; and (b) a sequence comprising SEQ ID NO:1; wherein the mts-siRNA target element is operably linked to a heterologous transcribable DNA polynucleotide. By “heterologous transcribable DNA polynucleotide,” it is meant that the transcribable DNA polynucleotide is heterologous with respect to the polynucleotide sequence to which it is operably linked. In specific embodiments, the recombinant DNA polynucleotide comprises an mts-siRNA target element sequence having at least about 90 percent, at least 91 percent, at least 92 percent, at least 93 percent, at least 94 percent, at least 95 percent, at least 96 percent, at least 97 percent, at least 98 percent, or at least 99 percent sequence identity to the DNA sequence of SEQ ID NOs:1. In some embodiments, the recombinant DNA polynucleotide comprises an mts-siRNA target element sequence comprising SEQ ID NOs:1 or a fragment thereof capable of binding an mts-siRNA.

In some embodiments, the heterologous transcribable DNA polynucleotide comprises a gene of agronomic interest. In one embodiment, the gene of agronomic interest confers pest resistance in plants. In another embodiment, the gene of agronomic interest confers herbicide tolerance in plants.

Several embodiments described herein relate to transgenic plant cells comprising an mts-siRNA target element comprising a DNA sequence selected from the group consisting of: (a) a sequence with at least 90 percent sequence identity to SEQ ID NOs:1; and (b) a sequence comprising SEQ ID NOs:1; wherein the mts-siRNA target element is operably linked to a heterologous transcribable DNA polynucleotide. In certain embodiments, the transgenic plant cell is a corn plant cell.

In another aspect, further provided herein is a transgenic plant, or part thereof, comprising an mts-siRNA target element comprising a DNA sequence selected from the group consisting of: (a) a sequence with at least 90 percent sequence identity to SEQ ID NOs:1; and (b) a sequence comprising SEQ ID NOs:1; wherein the mts-siRNA target element is operably linked to a heterologous transcribable DNA polynucleotide. In specific embodiments, the transgenic plant is a progeny plant of any generation that comprises the recombinant DNA polynucleotide. A transgenic seed comprising the recombinant DNA polynucleotide that produces such a transgenic plant when grown is also provided.

Several embodiments described herein relate to a method of producing a commodity product comprising obtaining a transgenic plant or part thereof containing a recombinant DNA polynucleotide as described herein and producing the commodity product therefrom. In one embodiment, the commodity product is selected from the group consisting of seeds, processed seeds, protein concentrate, protein isolate, starch, grains, plant parts, seed oil, biomass, flour, and meal.

Several embodiments provide a method of producing a transgenic plant comprising a recombinant DNA polynucleotide of comprising one or more mts-siRNA target elements as described herein comprising transforming a plant cell with the recombinant DNA polynucleotide to produce a transformed plant cell and regenerating a transgenic plant from the transformed plant cell.

Several embodiments provide a method of reducing expression of a transcribable DNA polynucleotide in male tissues of a plant comprising: (a) operably linking an mts-siRNA target element to said transcribable DNA polynucleotide; and (b) transforming a plant with an expression cassette comprising the transcribable DNA polynucleotide operably linked to the mts-siRNA target element; wherein the mts-siRNA target element has at least about 90 percent, at least 91 percent, at least 92 percent, at least 93 percent, at least 94 percent, at least 95 percent, at least 96 percent, at least 97 percent, at least 98 percent, at least 99 percent or 100% sequence identity to the DNA sequence of SEQ ID NO:1.

### DESCRIPTION OF THE FIGURES

FIG. 1 is a diagram illustrating an expression cassette comprised of a promoter and leader, operably linked 5′ to an intron, operably linked 5′ to a gene of interest (GOI), operably linked 5′ to an mts-siRNA target element, operably linked 5′ to a 3′ UTR. Transcription of the expression cassette results in an mRNA that comprises the mts-siRNA target element. A single-strand of double-stranded mts-siRNA can bind to a region within the mts-siRNA target element, triggering sequence-dependent endonucleolytic cleavage of the mRNA.

FIG. 2 is a graphical representation of the expression levels of the mts-siRNAs used to make mts-siRNA_TE_2 (SEQ ID NOs:3, 6, 2, 7, 5, 8, and 4) used as Cy5-labled probes to probe a transcript profiling microarray comprising cDNA from specific tissues of corn variety LH244.

### BRIEF DESCRIPTION OF THE SEQUENCES

SEQ ID NO:1 is a DNA sequence of a recombinant, chimeric mts-siRNA target element, mts-siRNA_TE_2.

SEQ ID NO:2 is a DNA sequence of a male tissue-specific short interfering RNA (mts-siRNA), mts-siRNA_1.

SEQ ID NO:3 is a DNA sequence of an mts-siRNA, mts-siRNA_2.

SEQ ID NO:4 is a DNA sequence of an mts-siRNA, mts-siRNA_3.

SEQ ID NO:5 is a DNA sequence of an mts-siRNA, mts-siRNA_4.

SEQ ID NO:6 is a DNA sequence of an mts-siRNA, mts-siRNA_6.

SEQ ID NO:7 is a DNA sequence of an mts-siRNA, mts-siRNA_7.

SEQ ID NO:8 is a DNA sequence of an mts-siRNA, mts-siRNA_8.

### DETAILED DESCRIPTION OF THE INVENTION

Several embodiments relate to recombinant DNA polynucleotides, compositions, and methods for selectively regulating protein expression, for instance expression of a heterologous transcribable polynucleotide, in a male reproductive tissue of a transgenic plant and uses thereof.

In some embodiments, a recombinant DNA polynucleotide that includes a male tissue-specific small interfering RNA target element (mts-siRNA_TE) operably linked to a heterologous transcribable polynucleotide is provided. Such recombinant DNA polynucleotides are useful for selectively regulating the expression of a heterologous transcribable polynucleotide in a male reproductive tissue of a transgenic plant. Nucleic acid sequences can be provided as DNA or as RNA, as specified; disclosure of one necessarily defines the other, as is known to one of ordinary skill in the art. Furthermore, disclosure of a given nucleic acid sequence necessarily defines and includes the complement of that sequence, as is known to one of ordinary skill in the art.

Small interfering RNA (siRNA) is a class of RNA molecules of about 18-26 nucleotides (nt) in length (for example, 18, 19, 20, 21, 22, 23, 24, 25, or 26 nt). An siRNA sequence may be represented using the RNA nucleotide sequence consisting of guanine (G), cytosine (C), adenine (A), and uracil (U) or using the equivalent DNA nucleotide sequence of guanine (G), cytosine (C), adenine (A), and thymine (T). siRNA functions within RNA-induced silencing complexes (RISCs) to trigger the sequence specific degradation of messenger RNA (mRNA), which results in the disruption of the gene expression and down-regulation of the protein encoded by the gene.

A “male tissue-specific siRNA” or “mts-siRNA” is an siRNA enriched or specifically expressed in the male reproductive tissues(s) (for example, male inflorescence or pollen) of a plant thus having a male tissue-specific expression pattern. Male tissue-specific siRNA have been identified in plants and can be detected using techniques known in the art, such as low molecular weight northern analysis or transcriptome sequencing.

“Male tissue(s)” or “male reproductive tissue(s)” refers to components of the male reproductive system of a plant. Male tissue of corn plants for example, may comprise tassels, male inflorescence, and pollen.

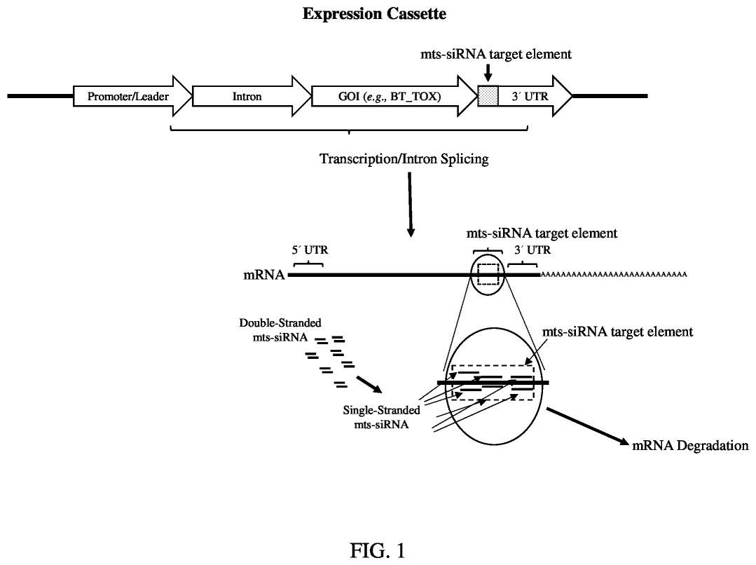

A DNA sequence that is complementary to an mts-siRNA sequence is referred to herein as an “mts-siRNA target”. An “mts-siRNA target element” comprises multiple sequences that are complementary to one or more mts-siRNA sequences. The mts-siRNA target element is contained in the DNA sequence of the gene of interest and is transcribed into the RNA sequence of the corresponding mRNA molecule. A single-strand of a double-stranded mts-siRNA molecule can then bind or hybridize under typical physiological conditions to a corresponding mts-siRNA target in an mRNA molecule (FIG. 1). A nucleic acid sequence is complimentary to an mts-siRNA sequence if an alignment of the two nucleic acid sequences produces an exact match (with no mismatches i.e., complete complement), one mismatch, two mismatches, or three mismatches over the length of the mts-siRNA sequence. Complementary sequences can base-pair with each other according to the standard Watson-Crick complementarity rules (e.g., guanine pairs with cytosine (G:C) and adenine pairs with either thymine (A:T) or uracil (A:U)).

More than one mts-siRNA target can be clustered together or even overlap within a single DNA polynucleotide. A DNA polynucleotide comprising more than one mts-siRNA targets is referred to herein as a “mts-siRNA target element”. An mts-siRNA target element comprises at least two or more mts-siRNA targets within a 500 nucleotide sequence window. An mts-siRNA target element can be any length, such as about 30 nucleotides (nt), about 40 nt, about 50 nt, about 60 nt, about 70 nt, about 80 nt, about 90 nt, about 100 nt, about 150 nt, about 200 nt, about 250 nt, about 300 nt, about 350 nt, about 400 nt, about 450 nt, or about 500 nt. An “mts-siRNA target sequence” is the nucleic acid sequence of an mts-siRNA target. An “mts-siRNA target element sequence” is the nucleic acid sequence of an mts-siRNA target element. An example of an mts-siRNA target element sequence is provided herein as SEQ ID NO:1.

FIG. 1 illustrates the effect of operably linking an mts-siRNA target element to a transcribable DNA polynucleotide in an expression cassette used to transform a plant. The mts-siRNA target element in this illustration is operably linked 3′ to a transcribable DNA polynucleotide denoted as a “GOI” and 5′ to a 3′ UTR. When expressed in the plant, si-RNAs expressed only in the male tissues of the plant (mts-siRNAs) that are complementary to regions of the mts-siRNA target element bind to the target element in the resulting transcript. The now double-stranded RNA segments of the resulting transcript trigger sequence-dependent endonucleolytic cleavage and degradation of the mRNA, thus reducing expression in the male tissues, for example, pollen and tassels of corn plants.

As used herein, a “recombinant” DNA polynucleotide, polypeptide, protein, cell, or organism may be a non-naturally occurring or man-made creation using the tools of genetic engineering and as such is the product of human activity and would not otherwise normally occur in nature. A “recombinant DNA polynucleotide” refers to a DNA polynucleotide comprising a combination of DNA sequences or polynucleotides that would not naturally occur together without human intervention. For instance, a recombinant DNA polynucleotide may be a DNA polynucleotide that is comprised of at least two DNA polynucleotides heterologous with respect to each other, a DNA polynucleotide that comprises a DNA sequence that deviates from DNA sequences that exist in nature, or a DNA polynucleotide that has been incorporated into a host cell's DNA by genetic transformation. In one embodiment, a recombinant DNA polynucleotide as described herein is a DNA polynucleotide comprising an mts-siRNA target element operably linked to at least one transcribable polynucleotide, for instance, where the transcribable polynucleotide is heterologous to the mts-siRNA target element.

As used herein, the term “heterologous” refers to the combination of two or more DNA sequences, RNA sequences, amino acid sequences, or protein domains when such a combination is not normally found in nature or when such a combination is provided in an orientation or order that is different than that found in nature. For example, the two DNA sequences may be derived from different species or created synthetically and/or the two DNA sequences may be derived from different genes, e.g., different genes from the same species or the same genes from different species. In one example, a regulatory element or mts-siRNA target element may be heterologous with respect to an operably linked transcribable polynucleotide sequence if such a combination is not normally found in nature, e.g., the transcribable polynucleotide sequence does not naturally occur operably linked to the regulatory element or mts-siRNA target element. In one embodiment, such a heterologous combination may comprise an mts-siRNA target element that may be plant-derived or chemically synthesized and may be operably linked to a transcribable polynucleotide, such as a transgene encoding a protein that confers insect resistance. In addition, a particular sequence can be “heterologous” with respect to a cell or organism into which it is introduced (for example, a sequence that does not naturally occur in that particular cell or organism).

As used herein, the term “transcribable DNA polynucleotide” refers to any DNA polynucleotide capable of being transcribed into an RNA polynucleotide, including, but not limited to, those having protein coding sequences, those encoding guide RNAs (gRNAs), and those producing RNA molecules having sequences useful for gene suppression (e.g., siRNAs, miRNAs, dsRNAs). The type of DNA polynucleotide can include, but is not limited to, a DNA polynucleotide from the same plant, a DNA polynucleotide from another plant, a DNA polynucleotide from a different organism, or a synthetic DNA polynucleotide, such as a DNA polynucleotide containing an antisense message of a gene, or a DNA polynucleotide encoding an artificial, synthetic, or otherwise modified version of a transgene. Examples of transcribable DNA polynucleotides for incorporation into constructs as described herein include, e.g., DNA polynucleotides or genes from a species other than the species into which the DNA polynucleotide is incorporated or genes that originate from, or are present in, the same species, but are incorporated into recipient cells by genetic engineering methods rather than classical breeding techniques.

A transcribable DNA polynucleotide may comprise a gene of agronomic interest. As used herein, the term “gene of agronomic interest” refers to a transcribable DNA polynucleotide that, when expressed in a particular plant tissue, cell, or cell type, confers a desirable characteristic. The product of a gene of agronomic interest may act within the plant in order to cause an effect upon the plant morphology, physiology, growth, development, yield, grain composition, nutritional profile, disease or pest resistance, and/or environmental or chemical tolerance or may act as a pesticidal agent in the diet of a pest that feeds on the plant. In one embodiment, a regulatory element is incorporated into a construct such that the regulatory element is operably linked to a transcribable DNA polynucleotide that is a gene of agronomic interest. In a transgenic plant containing such a construct, the expression of the gene of agronomic interest can confer a beneficial agronomic trait. A beneficial agronomic trait may include, for example, but is not limited to, insect protection, herbicide tolerance, modified yield, disease resistance, pathogen resistance, modified plant growth and development, modified starch content, modified oil content, modified fatty acid content, modified protein content, modified fruit ripening, enhanced animal and human nutrition, biopolymer productions, environmental stress resistance, pharmaceutical peptides, improved processing qualities, improved flavor, hybrid seed production utility, improved fiber production, augmented carbon sequestration, and/or desirable biofuel production. The mts-siRNA target element can be operably linked to the gene of agronomic interest to reduce the gene expression in male tissues such as tassel and pollen. In one embodiment, such a gene of agronomic interest may comprise a transgene encoding a protein that confers insect resistance.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

As used herein, the term “heterologous transcribable DNA polynucleotide,” refers to a transcribable DNA polynucleotide that is heterologous with respect to the mts-siRNA target element.

As used herein, the term “isolated” means separated from other elements typically associated with it in its natural state. For example, an isolated DNA polynucleotide is one that is present alone or in combination with other compositions but is not in its natural genomic location or state. In one embodiment, the term “isolated” refers to a DNA polynucleotide that is separated from the nucleic acids that normally flank the DNA polynucleotide in its natural state. For example, an isolated DNA polynucleotide may be a DNA polynucleotide that is comprised of at least two DNA sequences heterologous with respect to each other. In another example, an isolated DNA polynucleotide may be a DNA polynucleotide that has been incorporated into a novel genomic location in a host cell by genetic transformation. Thus, a DNA polynucleotide fused to or operably linked to one or more other DNA polynucleotide(s) with which it would not be associated in nature, for example as the result of recombinant DNA or plant transformation techniques, is considered isolated herein. Such polynucleotides are considered isolated even when integrated into the chromosome of a host cell or present in a nucleic acid solution with other DNA polynucleotides.

The term “operably linked” refers to at least two nucleotide sequences arranged or linked in a manner so that one can affect the function of the other. The two nucleotide sequences can be part of a single contiguous nucleotide molecule and can be adjacent or separated. For example, an mts-siRNA target element may be operably linked with a transcribable polynucleotide as shown in FIG. 1. In one embodiment, an operably linked mts-siRNA target element can affect the transcription, translation, or expression of the transcribable polynucleotide. For example, an mts-siRNA target element is operably linked to a transcribable polynucleotide if, after transcription in male reproductive tissue cell, the presence of the mts-siRNA target element in the mRNA molecule results in the regulation of the expression of the transcribable polynucleotide in the cell induced by the endogenous mts-siRNA and the RISC pathway. Operable linkage of the mts-siRNA target element and the transcribable polynucleotide can be achieved, for example, through incorporation of an mts-siRNA target element adjacent to the transcribable polynucleotide (such as located 5′ or 3′ to the transcribable polynucleotide, but not necessarily in contiguous linkage), in or adjacent to an untranslated region (UTR) of the polynucleotide (such as located in or next to the 5′ UTR or the 3′ UTR), and/or 3′ to the transcribable polynucleotide and 5′ to the polyadenylation signal. In one embodiment, an mts-siRNA target element is located between the transcribable polynucleotide and the polyadenylation sequence, that is 3′ to and adjacent to the transcribable polynucleotide. In another embodiment, an mts-siRNA target element is located between the stop codon of the transcribable polynucleotide and the polyadenylation sequence. In another embodiment, an mts-siRNA target element is located within the 3′ UTR sequence adjacent to the transcribable polynucleotide.

Examples of the identification of mts-siRNA sequences, mts-siRNA targets, and mts-siRNA target elements are provided herein and can be identified by methods known to those skilled in the art, for example through bioinformatic analysis of plant small RNA (sRNA) and complementary DNA (cDNA) libraries. In particular, mts-siRNA can be identified from sRNA libraries and sequenced. The identified mts-siRNA sequences can be compared to cDNA and/or genomic sequence collections to identify mts-siRNA targets and mts-siRNA target elements useful for developing recombinant DNA polynucleotides and constructs as described herein.

As used herein, the term “construct” means any recombinant DNA polynucleotide such as a plasmid, cosmid, virus, phage, or linear or circular DNA or RNA polynucleotide, derived from any source, capable of genomic integration or autonomous replication, comprising a DNA polynucleotide where at least one DNA polynucleotide has been linked to another DNA polynucleotide in a functionally operative manner, i.e. operably linked. As used herein, the term “vector” means any construct that may be used for the purpose of transformation, i.e., the introduction of heterologous DNA or RNA into a host cell. A construct typically includes one or more expression cassettes. As used herein, an “expression cassette” refers to a recombinant DNA polynucleotide comprising at least a transcribable DNA polynucleotide operably linked to one or more regulatory elements, typically at least a promoter and a 3′ UTR.

In some embodiments, mts-siRNA target elements are created, synthesized, or modified in vitro. For instance, mts-siRNA target elements may be modified to contain more, fewer, or different mts-siRNA target sequences or to rearrange the relative position of one or more mts-siRNA target sequence(s). In some embodiments, such modification may be beneficial in increasing or decreasing the effect of the mts-siRNA target element. Methods for creation, synthesis, or in vitro modification of an mts-siRNA target element and for determining the optimal variation for the desired level of regulation are known by those of skill in the art. For example, recombinant mts-siRNA target elements can be created by combining the DNA sequences, or fragments thereof, of SEQ ID NO:1, or one or more of the mts-siRNA sequences provided herein as SEQ ID NOs:2-8.

The DNA sequence of the mts-siRNA target element can also be varied by incorporating 1-3 nucleotide mismatches in an mts-siRNA target sequence (relative to a given mts-siRNA sequence). In another embodiment, the present invention includes variant recombinant DNA polynucleotide or mts-siRNA target elements having at least about 80% (percent) sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, and about 99% sequence identity to any of the recombinant mts-siRNA target elements (such as SEQ ID NOs:1-9). In certain embodiments, the variant recombinant DNA polynucleotide or mts-siRNA target elements have the activity of the base sequence from which they are derived.

Several embodiments relate to fragments of a DNA polynucleotide disclosed herein. Such fragments may be useful as mts-siRNA target elements or may be combined with other mts-siRNA targets, mts-siRNA target elements, mts-siRNA sequences, or fragments thereof for constructing recombinant mts-siRNA target elements, as described above. In specific embodiments, such fragments may comprise at least about 5, at least about 7, at least about 10, at least about 12, at least about 15, at least about 17, at least about 20, at least about 25, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 350, at least about 400, at least about 450, at least about 500 contiguous nucleotides, or longer, of a DNA polynucleotide disclosed herein, such as an mts-siRNA target element or cDNA sequence as disclosed herein. Methods for producing such fragments from a starting DNA polynucleotide are well known in the art. Fragments of recombinant DNA polynucleotide or mts-siRNA target elements as disclosed herein may have the activity of the base sequence from which they are derived.

The efficacy of the modifications, duplications, deletions, or rearrangements described herein on the desired expression aspects of a particular transcribable polynucleotide may be tested empirically in stable and transient plant assays, such as those described in the working examples herein, so as to validate the results, which may vary depending upon the changes made and the goal of the change in the starting DNA polynucleotide.

An mts-siRNA target and an mts-siRNA target element can function in either direction, meaning it is nondirectional, and as such can be used in either the 5′ to 3′ orientation or in the 3′ to 5′ orientation in a recombinant DNA polynucleotide or DNA construct.

As used herein, “expression of a transcribable polynucleotide” or “expression of a protein” refers to the production of a protein encoded by a transcribable polynucleotide and the resulting transcript (mRNA) in a cell. The term “protein expression” therefore refers to any pattern of translation of a transcribed RNA polynucleotide into a protein. Protein expression may be characterized by its temporal, spatial, developmental, or morphological qualities, as well as by quantitative or qualitative indications. In one embodiment, a recombinant DNA polynucleotide as described herein can be used to selectively regulate expression of a protein or transcribable polynucleotide in male reproductive tissues of a transgenic plant. In such an embodiment, expression of the recombinant DNA polynucleotide in a transgenic plant may result in expression of an operably linked transcribable polynucleotide in at least vegetative tissues but not in male reproductive tissues. In certain embodiments, such regulation of protein expression refers to suppressing or reducing; for example, suppressing or reducing the level of protein produced in a cell, for example through RNAi-mediated post-transcriptional gene regulation.

Selective regulation of protein expression as used herein refers to a reduction of protein production in a cell or tissue as compared to a reference cell or tissue by at least about 75%, at least about 80%, at least about 85%, at least about 90%, at least about 95%, at least about 99%, or 100% reduction (i.e., complete reduction). A reference cell or tissue can be, for example, a vegetative cell or tissue from the same or a similar transgenic plant expressing the protein or a cell or tissue from a transgenic plant comprising a similar transgene (or similar transcribable DNA polynucleotide) encoding the protein but lacking an operably linked mts-siRNA target element. Regulation of protein expression can be determined using any technique known to one skilled in the art, such as by directly measuring protein accumulation in a cell or tissue sample using a technique such as ELISA or western blot analysis, by measuring enzymatic activity of the protein, or by phenotypically determining protein expression. In one embodiment, selective regulation of protein expression refers to sufficient reduction in expression of a protein capable of conferring insect pest resistance in the male tissue of a transgenic plant. The reduction of protein in the male tissue such as pollen as measured by methods known in the art such as ELISA would therefore indicate the selective regulation of the protein expression.

As used herein, the term “transgene encoding a recombinant protein” or “transcribable polynucleotide” refers to any polynucleotide capable of being transcribed into an RNA molecule, including but not limited to, those having a nucleotide sequence encoding a polypeptide sequence. Depending upon conditions, the nucleotide sequence may or may not be actually translated into a polypeptide in a cell. The boundaries of a transgene or transcribable polynucleotide are commonly delineated by a translation start codon at the 5′-terminus and a translation stop codon at the 3′-terminus.

The term “transgene” refers to a DNA polynucleotide artificially incorporated into the genome of an organism or host cell, in the current or any prior generation of the organism or cell, as a result of human intervention, such as by plant transformation methods. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. In some embodiments, a transgenic plant comprises a recombinant DNA polynucleotide comprising one or more mts-siRNA target(s) as described herein.

In some embodiments, a transgene or transcribable polynucleotide includes, but is not limited to, a transgene or transcribable polynucleotide that provides a desirable characteristic associated with plant morphology, physiology, growth, development, yield, nutritional properties, disease resistance, pest resistance, herbicide tolerance, stress tolerance, environmental stress tolerance, or chemical tolerance. In one embodiment, a transcribable polynucleotide encodes a protein that when expressed in a transgenic plant confers insect pest resistance at least in a cell and/or tissue where the expressed protein is present; selective regulation of the insect pest toxin protein in male reproductive tissue of the transgenic plant results in at least reduced insect toxin protein expression in the pollen, thus protecting non-target insects that do not feed on the plant but may come in contact with the plant pollen from harm imparted by the toxin protein.

In some embodiments, a recombinant DNA construct as described herein may be made by techniques known in the art and in various embodiments are included in plant transformation vectors, plasmids, or plastid DNA. Such recombinant DNA constructs are useful for producing transgenic plants and/or cells and as such can also be contained in the genomic DNA of a transgenic plant, seed, cell, or plant part. In some embodiments, the recombinant DNA construct is located within a plant transformation vector, or on a biolistic particle for transforming a plant cell, or within a chromosome or plastid of a transgenic plant cell, or within a transgenic cell, transgenic plant tissue, transgenic plant seed, transgenic pollen grain, or a transgenic or partially transgenic (for example, a grafted) plant. A vector is any DNA polynucleotide that may be used for the purpose of plant transformation, i.e., the introduction of DNA into a plant cell. Recombinant DNA constructs, for example, can be inserted into a plant transformation vector and used for plant transformation to produce transgenic plants, seeds, and cells. Methods for constructing plant transformation vectors are well known in the art. Plant transformation vectors generally include but are not limited to: a suitable promoter for the expression of operably linked DNA, an operably linked recombinant DNA polynucleotide, and a polyadenylation signal (which may be included in a 3′ UTR sequence). Promoters useful in DNA constructs as described herein include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional optional components include, but are not limited to, one or more of the following targets: 5′ UTR, enhancer, cis-acting target, intron, signal sequence, transit peptide sequence, and one or more selectable marker genes. In one embodiment, a plant transformation vector comprises a recombinant DNA construct.

The recombinant DNA constructs and plant transformation vectors described herein may be made by any method suitable to the intended application, taking into account, for example, the type of expression desired, the transgene or transcribable polynucleotide desired, and convenience of use in the plant in which the recombinant DNA construct is to be expressed. General methods useful for manipulating DNA polynucleotides for making and using recombinant DNA constructs and plant transformation vectors are well known in the art and described in detail in, for example, handbooks and laboratory manuals including Michael R. Green and Joseph Sambrook, “Molecular Cloning: A Laboratory Manual” (Fourth Edition) ISBN:978-1-936113-42-2, Cold Spring Harbor Laboratory Press, NY (2012).

Recombinant DNA polynucleotides, constructs and plant transformation vectors as described herein can be modified by methods known in the art, either completely or in part, for example, for increased convenience of DNA manipulation (such as restriction enzyme recognition sites or recombination-based cloning sites), or for including plant-preferred sequences (such as plant-codon usage or Kozak consensus sequences), or to include sequences useful for recombinant DNA polynucleotide and construct design (such as spacer or linker sequences). In certain embodiments, the DNA sequence of the recombinant DNA polynucleotide and construct includes a DNA sequence that has been codon-optimized for the plant in which the recombinant DNA polynucleotide or construct is to be expressed. For example, a recombinant DNA polynucleotide or construct to be expressed in a plant can have all or parts of its sequence codon-optimized for expression in a plant by methods known in the art. The recombinant DNA polynucleotides or constructs as described herein can be stacked with other recombinant DNA polynucleotides or transgenic events for imparting additional traits (for example, in the case of transformed plants, traits including herbicide resistance, pest resistance, cold germination tolerance, water deficit tolerance) for example, by expressing or regulating other genes.

Several embodiments relate to transgenic plant cells, transgenic plant tissues, and transgenic plants or seeds that include a recombinant DNA polynucleotide as described herein. A further aspect includes artificial or recombinant plant chromosomes that include a recombinant DNA polynucleotide as described herein. Suitable methods for transformation of host plant cells for use with the recombinant DNA polynucleotide as described herein include virtually any method by which DNA can be introduced into a cell (for example, where a recombinant DNA polynucleotide is stably integrated into a plant chromosome) and are well known in the art. An exemplary and widely utilized method for introducing a recombinant DNA polynucleotide into plants is the Agrobacterium transformation system, which is well known to those of skill in the art. Another exemplary method for introducing a recombinant DNA polynucleotide into plants is insertion of a recombinant DNA polynucleotide into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example a CRISPR/Cas12a system). Transgenic plants can be regenerated from a transformed plant cell by the methods of plant cell culture. A transgenic plant homozygous with respect to a transgene can be obtained by sexually mating (selfing) an independent segregant transgenic plant that contains a single exogenous gene sequence to itself, for example a R₀ or F₀ plant, to produce R₁ or F₁ seed. One fourth of the R₁ or F₁ seed produced will be homozygous with respect to the transgene. Plants grown from germinating R₁ or F₁ seed can be tested for heterozygosity, typically using a SNP assay or a thermal amplification assay that allows for the distinction between heterozygotes and homozygotes (i.e., a zygosity assay).

Several embodiments relate to a transgenic plant having in its genome a recombinant DNA polynucleotide as described herein, including, without limitation, alfalfa, cotton, corn (also referred to as maize), canola, rice, soybean, and wheat, among others. In one embodiment, the transgenic plant having in its genome a recombinant DNA polynucleotide as described herein may be corn. Several embodiments relate to transgenic plant cells, plant parts, and progeny of such a transgenic plant. As used herein “progeny” includes any plant, seed, plant cell, and/or plant part produced from or regenerated from a plant, seed, plant cell, and/or plant part that included a recombinant DNA polynucleotide as described herein. Transgenic plants, cells, parts, progeny plants, and seeds produced from such plants can be homozygous or heterozygous for the recombinant DNA polynucleotide as described herein. In some embodiments, plant parts may include, but are not limited to, leaves, stems, roots, seeds, endosperm, ovule, and pollen. Plant parts may be viable, nonviable, regenerable, or non-regenerable. Several embodiments relate to transformed plant cells comprising a DNA polynucleotide as described herein. In some embodiments, transformed or transgenic plant cells include regenerable and nonregenerable plant cells.

In several embodiments, a recombinant DNA polynucleotide as described herein is in a commodity product produced from a transgenic plant, seed, or plant part; such commodity products include, but are not limited to, harvested parts of a plant, crushed or whole grains or seeds of a plant, or any food or non-food product comprising a recombinant DNA polynucleotide as described herein.

Several embodiments relate to a recombinant DNA polynucleotide comprising an mts-siRNA target element comprising a sequence with at least about 90 percent, at least about 91 percent, at least about 92 percent, at least about 93 percent, at least about 94 percent, at least about 95 percent, at least about 96 percent, at least about 97 percent, at least about 98 percent, at least about 99 percent sequence identity or more to SEQ ID NO:1 operably linked to a heterologous transcribable DNA polynucleotide. The mts-siRNA target element can be operably linked to the heterologous transcribable DNA polynucleotide during cloning of the expression cassette that will be transformed into the plant. Alternatively, the mts-siRNA target element can also be introduced and operably linked to a heterologous transcribable DNA polynucleotide within a transgenic plant through site-specific genome editing. Several methods for editing are known in the art involving different sequence-specific genome modification enzymes (or complexes of proteins and/or guide RNA) that modify the genomic DNA. In some embodiments, a site-specific genome modification enzyme modifies the genome by inducing a double-strand break (DSB) or nick at a desired genomic site or locus. In some embodiments, a site-specific genome modification enzyme modifies the genome by inducing deamination of one or more nucleotides at a desired genomic site or locus. In some embodiments, during the process of repairing the DSB or nick introduced by the genome modification enzyme, a donor template DNA such as that comprising an mts-siRNA target element may become integrated into the genome at the site of the DSB or nick. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases, reverse transcriptases and any combination thereof. In certain embodiments, a CRISPR system (e.g., a CRISPR/Cas9 system, a CRISPR/Cas12a system, etc.) can be utilized for targeting insertion of a blunt-end double-stranded DNA fragment comprising an mts-siRNA target element into a genomic target site comprising a heterologous transcribable DNA polynucleotide. In some embodiments, the CRISPR-Cas system is selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. CRISPR-Cas effector proteins of the CRISPR-Cas systems include, but are not limited to, Cas9, C2c1, C2c3, C2c4, C2c5, C2c8, C2c9, C2c10, Cas12a (also referred to as Cpf1), Cas12b, Cas12c, Cas12d, Cas12e, Cas12h, Cas12i, Cas12g, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5, Cas14a, Cas14b, and Cas14c effector protein.

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms and methods in molecular biology may also be found in Clark et al., Molecular Biology, Third Edition, Academic Press, Elsevier Inc., 2019; Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al., A Dictionary of Genetics, 6th ed., Oxford University Press: New York, 15 2247; and Lewin, Genes IX, Oxford University Press: New York, 2007.

### EMBODIMENTS

For further illustration, additional exemplary, non-limiting embodiments of the present disclosure are set forth below.

A first embodiment relates to a recombinant DNA polynucleotide comprising an mts-siRNA target element comprising a DNA sequence selected from the group consisting of: a.) a sequence with at least 90 percent sequence identity to SEQ ID NO:1; b.) a sequence comprising SEQ ID NO:1; and c.) a fragment of SEQ ID NO:1 that is capable of binding its corresponding mts-siRNA; wherein the mts-siRNA target element is operably linked to a heterologous transcribable DNA polynucleotide.

A second embodiment relates to the recombinant DNA polynucleotide of embodiment 1, wherein said sequence has at least 95 percent sequence identity to the DNA sequence of SEQ ID NO:1.

A third embodiment relates to the recombinant DNA polynucleotide of embodiment 1, wherein the heterologous transcribable DNA polynucleotide comprises a gene of agronomic interest.

A fourth embodiment relates to the recombinant DNA polynucleotide of embodiment 3, wherein the gene of agronomic interest confers pest resistance in plants.

A fifth embodiment relates to the recombinant DNA polynucleotide of embodiment 3, wherein the gene of agronomic interest confers herbicide tolerance in plants.

A sixth embodiment relates to the recombinant DNA polynucleotide of embodiment 3, wherein the gene of agronomic interest confers an alteration of architecture in plants.

A seventh embodiment relates to the recombinant DNA polynucleotide of embodiment 3, wherein the gene of agronomic interest confers an improvement of yield in plants.

An eight embodiment relates to a transgenic plant cell comprising a recombinant DNA polynucleotide comprising an mts-siRNA target element comprising a DNA sequence selected from the group consisting of: a.) a sequence with at least 90 percent sequence identity to SEQ ID NO:1; b.) a sequence comprising SEQ ID NO:1; and c.) a fragment of SEQ ID NO:1 that is capable of binding its corresponding mts-siRNA; wherein the mts-siRNA target element is operably linked to a heterologous transcribable DNA polynucleotide.

A nineth embodiment relates to the transgenic plant cell of embodiment 8, wherein said transgenic plant cell is a corn plant cell.

A tenth embodiment relates to a transgenic plant, or part thereof, comprising the recombinant DNA polynucleotide of embodiment 1.

An eleventh embodiment relates to a progeny plant of the transgenic plant of embodiment 10, or part thereof, wherein the progeny plant or part thereof comprises the recombinant DNA polynucleotide of embodiment 1.

A twelfth embodiment relates to a transgenic seed, wherein the seed comprises the recombinant DNA polynucleotide of embodiment 1.

A thirteenth embodiment relates to a method of producing a commodity product comprising obtaining a transgenic plant or part thereof according to embodiment 10 and producing the commodity product therefrom.

A fourteenth embodiment relates to the method of embodiment 13, wherein the commodity product is seeds, processed seeds, protein concentrate, protein isolate, starch, grains, plant parts, seed oil, biomass, flour, and meal.

A fifteenth embodiment relates to a method of expressing a transcribable DNA polynucleotide comprising obtaining a transgenic plant according to embodiment 10 and cultivating said plant, wherein the transcribable DNA polynucleotide is expressed.

A sixteenth embodiment relates to a method of reducing expression of a transcribable DNA polynucleotide in male tissues of a plant comprising: a.) operably linking an mts-siRNA target element to said transcribable DNA polynucleotide; and b.) transforming a plant with an expression cassette comprising the transcribable DNA polynucleotide operably linked to the mts-siRNA target element; wherein the mts-siRNA target element has at least 90 percent sequence identity to SEQ ID NO:1 or a fragment thereof.

A seventeenth embodiment relates to the method of embodiment 16, wherein the mts-siRNA is 100% identical to the DNA sequence of SEQ ID NO:1.

An eighteenth embodiment relates to the method of embodiment 16, wherein the mts-siRNA has at least 95 percent sequence identity to the DNA sequence of SEQ ID NO:1.

Embodiment 19 is a recombinant DNA polynucleotide comprising an mts-siRNA target element comprising a DNA sequence selected from the group consisting of:
a. a sequence with at least 90 percent sequence identity to SEQ ID NO:1; and
b. a sequence comprising SEQ ID NO:1,
   wherein the mts-siRNA target element is operably linked to a heterologous transcribable DNA polynucleotide.

Embodiment 20 is the recombinant DNA polynucleotide of embodiment 19, wherein the DNA sequence of the mts-siRNA target element has at least 95 percent sequence identity to the DNA sequence of SEQ ID NO:1.

Embodiment 21 is the recombinant DNA polynucleotide of any one of embodiments 19 or 20, wherein the DNA sequence of the mts-siRNA target element has at least 98 percent sequence identity to the DNA sequence of SEQ ID NO:1.

Embodiment 22 is the recombinant DNA polynucleotide of any one of embodiments 19 to 21, wherein the DNA sequence of the mts-siRNA target element has at least 99 percent sequence identity to the DNA sequence of SEQ ID NO:1.

Embodiment 23 is the recombinant DNA polynucleotide of any one of embodiments 19 to 22, wherein the heterologous transcribable DNA polynucleotide comprises a gene of agronomic interest.

Embodiment 24 is the recombinant DNA polynucleotide of embodiment 23, wherein the gene of agronomic interest confers pest resistance in plants.

Embodiment 25 is the recombinant DNA polynucleotide of embodiment 23, wherein the gene of agronomic interest confers herbicide tolerance in plants.

Embodiment 26 is the recombinant DNA polynucleotide of embodiment 23, wherein the gene of agronomic interest confers an alteration of architecture in plants.

Embodiment 27 is the recombinant DNA polynucleotide of embodiment 23, wherein the gene of agronomic interest confers an improvement of yield in plants.

Embodiment 28 is a transgenic plant cell comprising a recombinant DNA polynucleotide comprising an mts-siRNA target element comprising a DNA sequence selected from the group consisting of:
a. a sequence with at least 90, or at least 95, or at least 98, or at least 99 percent sequence identity to SEQ ID NO:1; and
b. a sequence comprising SEQ ID NO:1;
   wherein the mts-siRNA target element is operably linked to a heterologous transcribable DNA polynucleotide.

Embodiment 29 is the transgenic plant cell of embodiment 28, wherein said transgenic plant cell is a corn plant cell.

Embodiment 30 is a transgenic plant, or part thereof, comprising the recombinant DNA polynucleotide of any one of embodiments 19 to 27.

Embodiment 31 is a progeny plant of the transgenic plant of embodiment 30, or part thereof, wherein the progeny plant or part thereof comprises the recombinant DNA polynucleotide of any one of embodiments 19 to 27.

Embodiment 32 is the transgenic plant, or part thereof of embodiment 30 or the progeny plant, or part thereof of embodiment 31, wherein the plant is a corn plant.

Embodiment 33 is a transgenic seed, wherein the seed comprises the recombinant DNA polynucleotide of any one of embodiments 19 to 27.

Embodiment 34 is the transgenic seed of embodiment 33, wherein the seed is a corn seed.

Embodiment 35 is a method of producing a commodity product comprising obtaining a transgenic plant or part thereof according to embodiment 30 and producing the commodity product therefrom.

Embodiment 36 is the method of embodiment 35, wherein the commodity product is selected from the group consisting of seeds, processed seeds, protein concentrate, protein isolate, starch, grains, plant parts, seed oil, biomass, flour, and/or meal.

Embodiment 37 is a method of expressing a transcribable DNA polynucleotide comprising obtaining a transgenic plant according to embodiment 30 and cultivating said plant, wherein the transcribable DNA polynucleotide is expressed.

Embodiment 38 is a method of reducing expression of a transcribable DNA polynucleotide in male reproductive tissue of a plant comprising the steps:
a. operably linking an mts-siRNA target element to said transcribable DNA polynucleotide, wherein said mts-siRNA target element is heterologous with respect to the transcribable DNA polynucleotide; and wherein the mts-siRNA target element has at least 90 percent sequence identity to SEQ ID NO:1; and
b. transforming a plant with an expression cassette comprising the transcribable DNA polynucleotide operably linked to the mts-siRNA target element.

Embodiment 39 is the method of embodiment 38, further comprising step
c. expressing the transcribable DNA polynucleotide comprising obtaining a transgenic plant from step b. and cultivating said plant, wherein the transcribable DNA polynucleotide is expressed.

Embodiment 40 is the method of embodiment 39, further comprising step
d. assaying the plant for reduced expression of the transcribable DNA polynucleotide in male reproductive tissue comprising
   (i) determining the level of expression of the protein encoded by the transcribable DNA polynucleotide in male reproductive tissue in the plant obtained from step c., and
   (ii) determining the level of expression of the protein encoded by the transcribable DNA polynucleotide in male reproductive tissue of a reference cell or tissue,
wherein the reference cell or tissue is from the same or a similar transgenic plant expressing said protein but lacking the operably linked mts-siRNA target element; or
wherein the reference cell or tissue is from a transgenic plant comprising a similar transcribable DNA polynucleotide encoding said protein but lacking the operably linked mts-siRNA target element.

Embodiment 41 is the method of any one of embodiments 38 to 40, wherein the mts-siRNA target element has at least 95 percent sequence identity to the DNA sequence of SEQ ID NO:1.

Embodiment 42 is the method of any one of embodiments 38 to 41, wherein the mts-siRNA target element has at least 98 percent sequence identity to the DNA sequence of SEQ ID NO:1.

Embodiment 43 is the method of any one of embodiments 38 to 42, wherein the mts-siRNA target element has at least 99 percent sequence identity to the DNA sequence of SEQ ID NO:1.

Embodiment 44 is the method of any one of embodiments 38 to 43, wherein the mts-siRNA target element comprises a DNA sequence comprising SEQ ID NO:1.

Embodiment 45 is the method of any one of embodiments 38 to 44, wherein the plant is a corn plant.

Embodiment 46 is the method of any one of embodiments 38 to 45, wherein the male reproductive tissue is pollen.

Embodiment 47 is the method of any one of embodiments 38 to 46, wherein the transcribable DNA polynucleotide comprises a gene of agronomic interest encoding a protein conferring pest resistance in plants or herbicide tolerance in plants.

Embodiment 48 is an isolated recombinant DNA molecule, characterized by comprising a DNA sequence of SEQ ID NO:1, wherein said DNA sequence is operably linked to a heterologous transcribable DNA molecule.

Embodiment 49 is the isolated recombinant DNA molecule of embodiment 48, characterized in that the heterologous transcribable polynucleotide molecule comprises a gene of agronomic interest.

Embodiment 50 is the isolated recombinant DNA molecule of embodiment 49, characterized in that the gene of agronomic interest confers herbicide tolerance in plants.

Embodiment 51 is the isolated recombinant DNA molecule of embodiment 49, characterized in that the gene of agronomic interest confers pest resistance in plants.

Embodiment 52 is a method of producing a transgenic plant, excluding the plant obtained by said method, characterized by comprising:
a. transforming a plant cell with the isolated recombinant DNA molecule of embodiment 48 to produce a transformed plant cell; and
b. regenerating a transgenic plant from the transformed plant cell.

Embodiment 53 is a construct characterized by comprising the isolated recombinant DNA molecule of embodiment 48.

The embodiments described herein may be more readily understood through reference to the following examples, which are provided by way of illustration, and are not intended to be limiting, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples are included to demonstrate embodiments of the disclosure. It should be appreciated by those of skill in the art that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1

### Identification and Synthesis of Mts-siRNA_TE_2

This example describes the identification of mts-siRNA sequences that were used to construct the mts-siRNA target element mts-siRNA_TE_2 (SEQ ID NO:1).

Small RNA libraries were prepared from leaf and pollen tissues derived from corn varieties B73 and LH244 using the TruSeq® Small RNA library Prep kit (Illumina, San Diego, CA) following the manufacturer's protocol. After cDNA amplification, uniquely indexed libraries were pooled together in equimolar amounts and size separated on a 6% Novex™ TBE Page gel (Novex, Fairfield, NJ). The gel was stained for twenty minutes with 1×SYBR Gold. The size-fractionated indexed cDNAs were then isolated and sequenced. A minimum of 3 million reads per size-selected library was generated using Illumina's NextSeq 550 platform. Bioinformatic analysis was used to compare the sequences between the leaf and pollen libraries to identify small RNAs that were only expressed in pollen (mts-siRNA). Table 1 shows the male tissue-specific small interfering RNAs and their corresponding SEQ ID NOs that were used in synthesizing mts-siRNA_TE_2.

TABLE 1
Male tissue-specific small interfering RNAs
and corresponding SEQ ID NOs.

| | SEQ | | |
|---|---|---|---|
| | ID | | Length |
| mts-siRNA | NO: | Sequence | (bp) |
| mts-siRNA_1 | 2 | ATGAAGCCGATTGTGGACATCGAT | 24 |
| mts-siRNA_2 | 3 | TGACCTCCATGGCAGTAGGATGCA | 24 |
| mts-siRNA_3 | 4 | TTCCACCAGGCATTTTCGTCCAAG | 24 |
| mts-siRNA_4 | 5 | AGTCGGCACAATGTACAGGTAGT | 23 |
| mts-siRNA_6 | 6 | TGATTGCGTAGAGAGCCCTTGGTT | 24 |
| mts-siRNA_7 | 7 | ACTACAGTGAGAGTCTAGGAAAGC | 24 |
| mts-siRNA_8 | 8 | CTCGGCGTCACTCACAACTAAC | 22 |

The mts-siRNA target element was chemically synthesized. The mts-siRNA target element, mts-siRNA_TE_2 (SEQ ID NO:1) was comprised of the mts-siRNA sequences presented in Table 1 above; four of which were placed in the reverse compliment (RC) orientation. Table 2 below shows the order of the SEQ ID NOs used to construct mts-siRNA_TE_2. Those mts-siRNA sequences indicated as “(RC)” are present in the reverse compliment orientation.

TABLE 2
mts-siRNA sequences comprised within
the mts-siRNA target elements.

| mts-siRNA Target | SEQ | | Length |
|---|---|---|---|
| Element | ID NO: | mts-siRNA SEQ ID NOs: | (bp) |
| mts-siRNA_TE_2 | 1 | 3(RC); 6; 2(RC); 7; 5(RC); 8; | 168 |
| | | 4(RC) | |

The mts-siRNA target element, mts-siRNA_TE_2 was cloned 3′ in operable linkage to an insect toxin coding sequence, BT_TOX. Transcription of the BT_TOX coding sequence would result in an mRNA that comprised a 5′ UTR, the BT_TOX coding sequence, an mts-siRNA target element, and a 3′ UTR (see FIG. 1).

### Example 2

### Efficacy of Mts-siRNA_TE_2 in Reducing Pollen Protein Expression

Corn plants were transformed with vectors, specifically plant expression vectors comprising an expression cassette using a constitutive promoter, leader, and intron driving expression of an insect toxin gene, BT_TOX which was operably linked to mts-siRNA_TE_2 to assess the efficacy of mts-siRNA_TE_2 in reducing expression of the toxin gene.

Corn plants were transformed with a binary plasmid plant transformation vector. The transformation vector was constructed using standard methods known in the art. The resulting plant transformation vector contained a left border region from Agrobacterium tumefaciens, a first expression cassette used to assess the efficacy of mts-siRNA_TE_2 (SEQ ID NO:1) comprised of a plant constitutive promoter, leader, and intron, operably linked 5′ to a coding sequence for the toxin, BT_TOX, operably linked 5′ to mts-siRNA_TE_2, operably linked 5′ to a 3′ UTR; a second expression cassette used for the selection of transformed plant cells using glyphosate selection; and a right border region from Agrobacterium tumefaciens.

Corn plant cells were transformed using the binary transformation vector constructs described above by an Agrobacterium-mediated transformation, as is well known in the art. The resulting transformed plant cells were induced to form whole corn plants.

Single copy stably transformed corn plants were selected for assay. BT_TOX protein expression was assayed in pollen using ELISA. Transformed corn plants comprising a BT_TOX expression cassette without an mts-siRNA target element were used as a control. Multiple R₀ transformants were assayed for mts-siRNA_TE_2 and the control. The expression levels of BT_TOX were assayed using ELISA in both leaf and pollen samples derived from both mts-siRNA_TE_2 and the control. Table 3 below shows the average expression of BT_TOX protein in leaf and pollen measured in parts per million (ppm) and percent reduced expression relative to the control.

TABLE 3
Average BT_TOX protein expression in leaf
and pollen, and percent reduced expression.

| | | | | Percent | Percent |
|---|---|---|---|---|---|
| | SEQ | | | Reduced | Reduced |
| | ID | Leaf | Pollen | Leaf | Pollen |
| mts-siRNA_TE | NO: | (ppm) | (ppm) | Expression | Expression |
| No mts- | — | 1166.3 | 90 | — | — |
| siRNA_TE | | | | | |
| mts-siRNA_{—} | 1 | 490.2 | 11.1 | 58% | 88% |
| TE_2 | | | | | |

As can be seen in Table 3, operable linkage of mts-siRNA_TE_2 (SEQ ID NO:1) was able to reduce BT_TOX protein expression in pollen.

### Example 3

### The Target Element, Mts-siRNA_TE_2 Binds Primarily to siRNAs Expressed in Male Corn Tissues

This example demonstrates that the siRNA sequences used to construct the target element, mts-siRNA_TE_2 are expressed primarily in the male tissues of corn such as young and old tassels as determined using Cy5-labeled probes hybridized to an siRNA micro-array.

Small RNAs were purified from ear, leaf, root, silk, and tassel tissues of corn varieties LH244 and O1DKD2 using the mirPremier® microRNA Isolation Kit (Sigma, St. Louis, MO). Small RNA libraries were made using the TruSeq® Small RNA library Prep kit (Illumina, San Diego, CA) following the manufacturer's protocol. The libraries were sequenced using Illumina's NextSeq 550 platform. Male tissue-specific short interfering RNAs (mts-siRNAs) were identified bioinformatically as those only expressed in tassel through a comparison of the library sequences from each tissue. Complementary sequences of 1,216 mts-siRNAs specific to tassel were arrayed in triplicate onto custom microarray chips produced by LC Sciences (Houston, TX). Total RNAs isolated from 26 wild-type tissues collected from 31 germplasms were used to synthesize complementary DNA (cDNA). Cy5-labelelled complimentary RNAs were then prepared from the cDNA samples and hybridized with the custom microarray chips. Hybridization images were collected using a GenePix1® 4000B laser scanner (Molecular Devices, Sunnyvale, CA) and digitized using Array-Pro® Analyzer image analysis software (Media Cybernetics, Rockville, MD). Relative signal values were derived by background subtraction and normalization. Differentially expressed signals were determined by t-test with p<0.05.

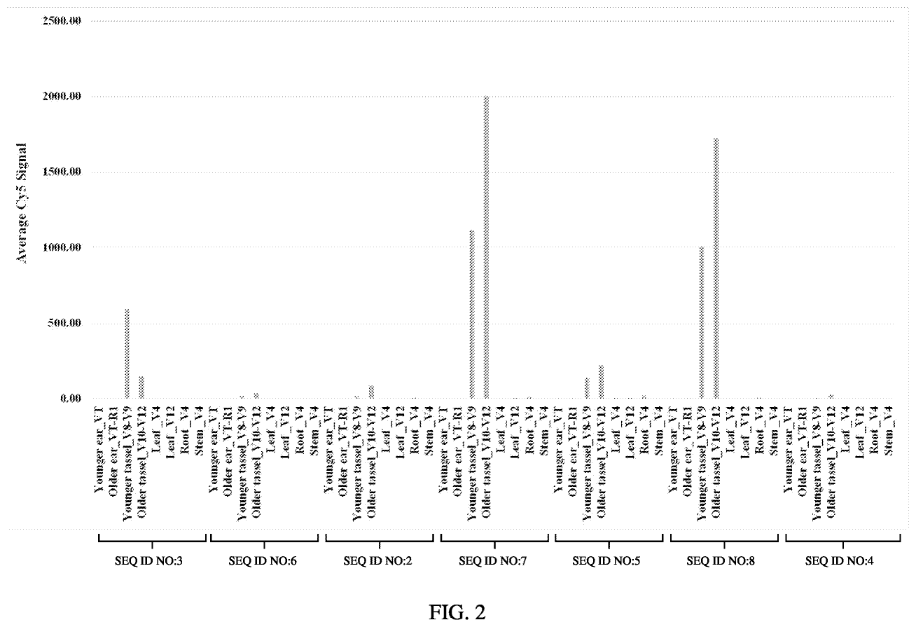

FIG. 2 shows the average Cy5 signal detected for the 7 mts-siRNA sequences used to construct mts-siRNA_TE_2 (SEQ ID NOs:3, 6, 2, 7, 5, 8, and 4) in various tissues of corn variety LH244. As can be seen in FIG. 2, expression of the mts-siRNAs used to construct mts-siRNA_TE_2 (SEQ ID NO:1) are expressed primarily in younger and older tassels. Very low expression was observed for SEQ ID NOs:7, 5, and 6 in V4 root.

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

1. A recombinant DNA polynucleotide comprising an mts-siRNA target element comprising a DNA sequence selected from the group consisting of:
i. a sequence with at least 90 percent sequence identity to SEQ ID NO:1; and
ii. a sequence comprising SEQ ID NO:1;
wherein the mts-siRNA target element is operably linked to a heterologous transcribable DNA polynucleotide.

2. The recombinant DNA polynucleotide of claim 1, wherein the DNA sequence of the mts-siRNA target element has at least 95 percent sequence identity to the DNA sequence of SEQ ID NO:1.

3. The recombinant DNA polynucleotide of claim 1, wherein the heterologous transcribable DNA polynucleotide comprises a gene of agronomic interest.

4. The recombinant DNA polynucleotide of claim 3, wherein the gene of agronomic interest confers pest resistance in plants.

5. The recombinant DNA polynucleotide of claim 3, wherein the gene of agronomic interest confers herbicide tolerance in plants.

6. The recombinant DNA polynucleotide of claim 3, wherein the gene of agronomic interest confers an alteration of architecture in plants.

7. The recombinant DNA polynucleotide of claim 3, wherein the gene of agronomic interest confers an improvement of yield in plants.

8. A transgenic plant cell comprising a recombinant DNA polynucleotide comprising an mts-siRNA target element comprising a DNA sequence selected from the group consisting of:
i. a sequence with at least 90 percent sequence identity to SEQ ID NO:1; and
ii. a sequence comprising SEQ ID NO:1;
wherein the mts-siRNA target element is operably linked to a heterologous transcribable DNA polynucleotide.

9. The transgenic plant cell of claim 8, wherein the transgenic plant cell is a corn plant cell.

10. A transgenic plant, or part thereof, comprising the recombinant DNA polynucleotide of claim 1.

11. A progeny plant of the transgenic plant of claim 10, or part thereof, wherein the progeny plant or part thereof comprises the recombinant DNA polynucleotide of claim 1.

12. A transgenic seed, wherein the seed comprises the recombinant DNA polynucleotide of claim 1.

13. A method of producing a commodity product comprising obtaining a transgenic plant or part thereof according to claim 10 and producing the commodity product therefrom.

14. The method of claim 13, wherein the commodity product is selected from the group consisting of seeds, processed seeds, protein concentrate, protein isolate, starch, grains, plant parts, seed oil, biomass, flour, and meal.

15. A method of expressing a transcribable DNA polynucleotide comprising obtaining a transgenic plant according to claim 10 and cultivating said plant, wherein the transcribable DNA polynucleotide is expressed.

16. A method of reducing expression of a transcribable DNA polynucleotide in male reproductive tissues of a plant comprising the steps:
i. operably linking an mts-siRNA target element to said transcribable DNA polynucleotide; wherein said mts-siRNA target element is heterologous with respect to the transcribable DNA polynucleotide; and wherein the mts-siRNA target element has at least 90 percent sequence identity to SEQ ID NO:1;
ii. transforming a plant with an expression cassette comprising the transcribable DNA polynucleotide operably linked to the mts-siRNA target element.

17. The method of claim 16, wherein the mts-siRNA target element comprises a DNA sequence comprising SEQ ID NO:1.

18. The method of claim 16, wherein the mts-siRNA target element has at least 95 percent sequence identity to the DNA sequence of SEQ ID NO:1.

19. The method of claim 16, further comprising step
c. expressing the transcribable DNA polynucleotide comprising obtaining a transgenic plant from step b. and cultivating said plant, wherein the transcribable DNA polynucleotide operably linked to the mts-siRNA target element is expressed.

20. The method of claim 16, wherein
i. the plant is a corn plant;
ii. the male reproductive tissue is pollen; and/or
iii. the transcribable DNA polynucleotide comprises a gene of agronomic interest encoding a protein conferring pest resistance in plants or herbicide tolerance in plants.


SEQUENCE LISTING (8 sequences)

SEQ ID NO: 1 - DNA, 168 bp, synthetic construct
  source 1..168: mol_type = other DNA; note = DNA sequence of a recombinant, chimeric mts-siRNA target element, mts-siRNA_TE_2.
gtatgcatcctactgccatggaggtcatgattgcgtagagagcccttggttatcgatgtccacaatcggcttcatactacagtgagagtctaggaaagcactacctgtacattgtgccgactctcggcgtcactcacaactaaccttggacgaaaatgcctggtggaa

SEQ ID NO: 2 - DNA, 24 bp, Zea mays
  source 1..24: mol_type = genomic DNA; note = DNA sequence of a male tissue-specific short interfering RNA (mts-siRNA), mts-siRNA_1.
atgaagccgattgtggacatcgat

SEQ ID NO: 3 - DNA, 24 bp, Zea mays
  source 1..24: mol_type = genomic DNA; note = DNA sequence of a mts-siRNA, mts-siRNA_2.
tgacctccatggcagtaggatgca

SEQ ID NO: 4 - DNA, 24 bp, Zea mays
  source 1..24: mol_type = genomic DNA; note = DNA sequence of a mts-siRNA, mts-siRNA_3.
ttccaccaggcattttcgtccaag

SEQ ID NO: 5 - DNA, 23 bp, Zea mays
  source 1..23: mol_type = genomic DNA; note = DNA sequence of a mts-siRNA, mts-siRNA_4.
agtcggcacaatgtacaggtagt

SEQ ID NO: 6 - DNA, 24 bp, Zea mays
  source 1..24: mol_type = genomic DNA; note = DNA sequence of a mts-siRNA, mts-siRNA_6.
tgattgcgtagagagcccttggtt

SEQ ID NO: 7 - DNA, 24 bp, Zea mays
  source 1..24: mol_type = genomic DNA; note = DNA sequence of a mts-siRNA, mts-siRNA_7.
actacagtgagagtctaggaaagc

SEQ ID NO: 8 - DNA, 22 bp, Zea mays
  source 1..22: mol_type = genomic DNA; note = DNA sequence of a mts-siRNA, mts-siRNA_8.
ctcggcgtcactcacaactaac